(19) United States
(12) Patent Application Publication
(10) Pub. No.: US 2003/0167018 A1
(43) Pub. Date: Sep. 4, 2003

(54) Sleep apnea device and method thereof

(76) Inventor: Robert Wyckoff, Smith River, CA (US)

Correspondence Address: MYERS & KAPLAN, INTELLECTUAL, PROPERTY LAW, L.L.C., 1827 POWERS FERRY ROAD, BUILDING 3, SUITE 200,, ATLANTA, GA, 30339, US

(21) Appl. No.: 10/090,268

(22) Filed: Mar. 4, 2002

(51) Int. Cl. A61B 5/08

(52) U.S. Cl. 600/538

(57) ABSTRACT

A neck-worn device and a method thereof, wherein a plate having a generally arcuate configuration is placed securely and removably on the neck of a user, wherein a substantially airtight zone is created between the device and the neck of a user, and wherein a valve is provided to allow the escape of air from the airtight zone in response to soft neck tissue respiratory movements, thus enabling the creation of a negative pressure or vacuum and thereby effectively drawing open the air passages of a user. The present invention is particularly suited for, although not limited to, utilization as a sleep apnea device enabling a user to alleviate the bothersome and potentially detrimental effects of sleep apnea without utilizing costly equipment requiring electrical or battery power.

### TECHNICAL FIELD

The present invention relates generally to air pathway clearance devices and, more specifically, to a neck-worn device and a method thereof, wherein a generally negative pressure is created on the exterior surface of a user's neck, thereby effectively holding open the air pathways. The present invention is particularly suited for, although not limited to, utilization as a sleep apnea device enabling a user to alleviate the bothersome and potentially detrimental effects of sleep apnea without utilizing costly equipment requiring electrical or battery power.

### BACKGROUND OF THE INVENTION

Sleep apnea affects millions of individuals, causing each to experience a variety of symptoms while sleeping. These symptoms often decrease feelings of restfulness and reduce health benefits derived from adequate rapid eye movement (rem) sleep sessions. While the degree of effect varies between individuals, most sleep apnea sufferers with obstructive sleep apnea experience collapse and closure of the soft tissues which form the anterior and lateral walls of the pharynx causing erratic cessations of natural breathing cycles and airflow, disruptive snoring behaviors and drops in oxygen saturation, potentially leading to periodic stoppages and/or interruptions of heart rhythms and blood flow, increased cardiovascular disease risk, hypertension, and in extreme cases, even death.

The most popular, presently available, non-surgical treatment method for sleep apnea relies on an electrical instrument, or Continuous Positive Airway Pressure (CPAP) machine. The basic premise behind the CPAP machine and its ability to counteract the affects of sleep apnea rests in the creation of a closed respiratory system for the user, wherein a generally constant and positive pressure forces the airways to remain open. This closed, positive pressure system utilizes a powered generator to blow a stream of air into the user's face through a mask typically worn over the users nose. The complexity of the electronic CPAP instrument makes the device costly to purchase, thus eliminating its availability to many sleep apnea sufferers.

Moreover, due to the significant expense of a CPAP machine and the need to calibrate the machine to a specific pressure, a complete diagnosis is desired, wherein the testing for such a diagnosis involves multiple visits to a sleep therapy clinic, or polysomnography laboratory. Such specialty evaluation is costly, as well as time consuming.

Even if an individual is successfully diagnosed and is financially able to obtain a CPAP machine, utilization of such an instrument presents ongoing disadvantages. For instance, machine operation produces noise. Moreover, the size of the machine hinders true portability and the necessity for electrical power presents insurmountable challenges in remote areas or in countries with non-adaptable power supply systems. In addition, wearing a face mask for sleeping is uncomfortable for many individuals, often creating painful pressure points on the face and nose. Moreover, the fixed setting, constant flow of cold, dry air into the face of the user can be bothersome, creating mucosal discomfort and promoting sinus and respiratory illnesses.

Therefore, it is readily apparent that there is a need for a non-complicated, inexpensive, truly portable and self-regulating sleep apnea device enabling a user to comfortably relieve the bothersome and potentially detrimental effects of sleep apnea, thus preventing the above-discussed disadvantages.

### BRIEF SUMMARY OF THE INVENTION

Briefly described, in a preferred embodiment, the present invention overcomes the above-mentioned disadvantages and meets the recognized need for such a device by providing a neck-worn device enabling a user to effectively address severe snoring problems and/or counteract the symptoms of sleep apnea by creating a negative pressure on the exterior surface of the neck, thereby drawing the neck out and holding air passages open.

According to its major aspects and broadly stated, the present invention is a plate having a generally arcuate configuration, wherein the arcuate configuration enables generally flush placement of the device against the neck of a user. Preferably adjustable straps enable secure positioning of the device around the neck of a user, wherein a seal is provided proximate to the peripheral edge of the device enabling formation of a substantially airtight zone between the device and the neck of a user, and wherein a valve is provided to allow the escape of air from the airtight zone when neck tissues impinge into it during exhalation, thus enabling the creation of a negative pressure or vacuum, thereby effectively drawing open the air passages of a user. In subsequent inhalation, the soft tissues are drawn inward and away from the airtight zone from which the ingress of air is occluded, thus further increasing the vacuum until further constriction of the airway is prevented. The more the pharyngeal air passage is narrowed, the greater the respiratory effort resulting in more expansion and contraction of the neck, wherein both movements increase the vacuum within the airtight zone to resolve the respiratory distress; thus, the device is self-regulating.

More specifically, the present invention is a neck worn device, wherein closure of the air passages of a user is essentially prevented. The device is generally shaped to conform with the exterior curvature of the neck, wherein a generally elongated member is arcuately-shaped and is worn on the front a user's neck.

At least one strap is provided on the support member, whereby secured positioning of the device on the neck of the user is enabled. The dimensions of the support member preferably enable comfortably secure placement along the mandibular area and on the clavicular area, wherein the support member preferably does not exert pressure upon the carotid body, carotid arties and/or jugular vein with nerve accompaniments, but extends beyond the endangered structures, preferably about ⅔ of the distance from the anterior to the posterior. Thus, the dimensions of the support member and the overall dimensions of the device preferably gener ally vary and correspond to the neck size of the intended user.

The support member has an interior surface and an exterior surface, wherein during use the interior surface is positioned proximate to the neck of the user. A seal is provided on the interior surface of the support member, proximate to the peripheral edge, wherein the seal extends outwardly from the interior surface of the support member and, when placed against the neck of a user, creates a substantially airtight zone between the support member and the neck of the user.

A vacuum valve defines a one-way passageway through the support member, thereby enabling the escape of air from the airtight zone following the expansion of the soft tissues of the neck during exhalation, thus enabling the creation of a partial vacuum or negative pressure as the same soft tissues are contracted by subsequent inhalation. As the tissues become increasingly contracted, inhalation increases the realized vacuum pressure effectively drawing open the air passages of a user.

A collection valve may also be provided, wherein a collection vessel may be connected thereto for analysis of performance.

A feature and advantage of the present invention is the ability of such a method and device to counteract the symptoms of sleep apnea by creating a negative pressure on the exterior surface of the neck.

A feature and advantage of the present invention is the ability of such a method and device to effectively draw open the air passageways of a user.

Another feature and advantage of the present invention is the ability of such a method and device to draw the neck out and hold air passages open.

Another feature and advantage of the present invention is the ability of such a method and device to be comfortably worn by a user without the creation of pressure points.

Another feature and advantage of the present invention is the ability of such a method and device to prevent the closure of air pathways.

Another feature and advantage of the present invention is the ability of such a method and device to adjust to a variety of individual sizes and shapes.

Another feature and advantage of the present invention is the ability of such a method and device to enable formation of a substantially airtight zone between the device and the neck of a user.

Another feature and advantage of the present invention is the ability of such a method and device to assist an individual in maintaining a clear air pathway.

Another feature and advantage of the present invention is the ability of such a method and device in its preferred form to enable a user to alleviate the bothersome and potentially detrimental effects of sleep apnea without utilizing costly equipment requiring electrical or battery power.

Another feature and advantage of the present invention is its ability to provide an uncomplicated, inexpensive and truly portable sleep apnea device and method of prevention.

Another feature and advantage of the present invention is the ability of such a method and device to enable a user to comfortably relieve the bothersome and potentially detrimental effects of sleep apnea.

Another feature and advantage of the present invention is the ability of such a method and device to provide automatic adjustment to the respiratory needs of a user.

Another feature and advantage of the present invention is the ability of such a method and device to self-regulate performance.

These and other objects, features and advantages of the invention will become more apparent to one skilled in the art from the following description and claims when read in light of the accompanying drawings.

### BRIEF DESCRIPTION OF THE DRAWINGS

The present invention will be better understood by reading the Detailed Description of the Preferred and Alternate Embodiments with reference to the accompanying drawing figures, in which like reference numerals denote similar structure and refer to like elements throughout, and in which:

FIG. 1 is a perspective view of a sleep apnea apparatus according to a preferred embodiment of the present invention.

FIG. 2 is a perspective view of the sleep apnea apparatus of FIG. 1, showing the interior surface of the support member.

FIG. 3 is a perspective view of the sleep apnea apparatus of FIG. 1, showing the apparatus being worn by an individual.

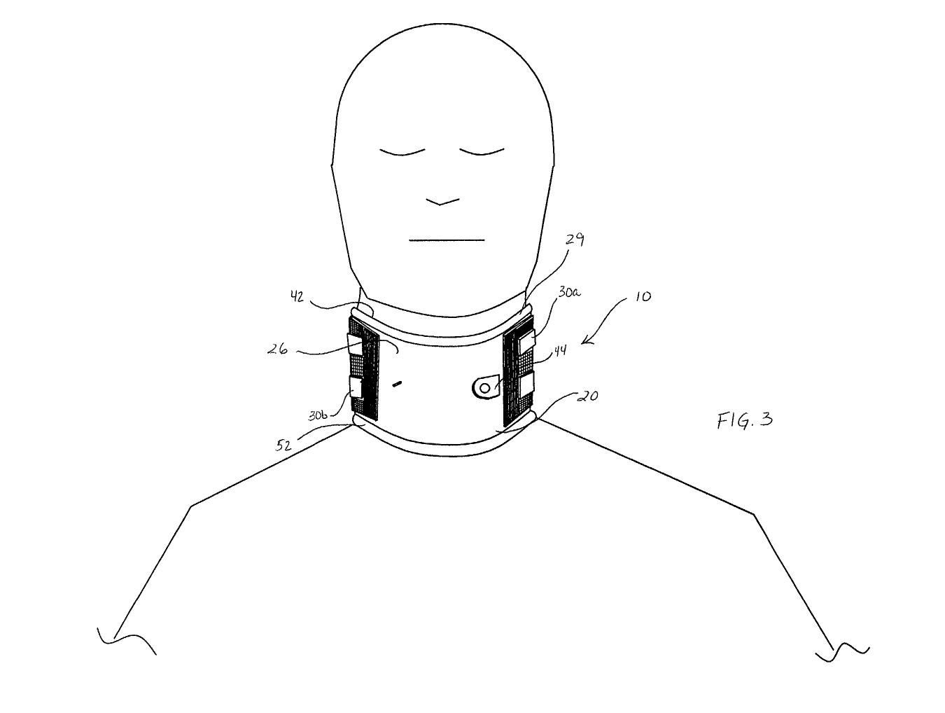

FIG. 4 is a perspective view of a sleep apnea apparatus according to an alternate embodiment of the present invention, showing the apparatus being worn by an individual and showing the apparatus connected to an external negative pressure means.

### DETAILED DESCRIPTION OF THE PREFERRED AND ALTERNATE EMBODIMENTS

In describing the preferred and alternate embodiments of the present invention, as illustrated in the figures and/or described herein, specific terminology is employed for the sake of clarity. The invention, however, is not intended to be limited to the specific terminology so selected, and it is to be understood that each specific element includes all technical equivalents that operate in a similar manner to accomplish similar functions.

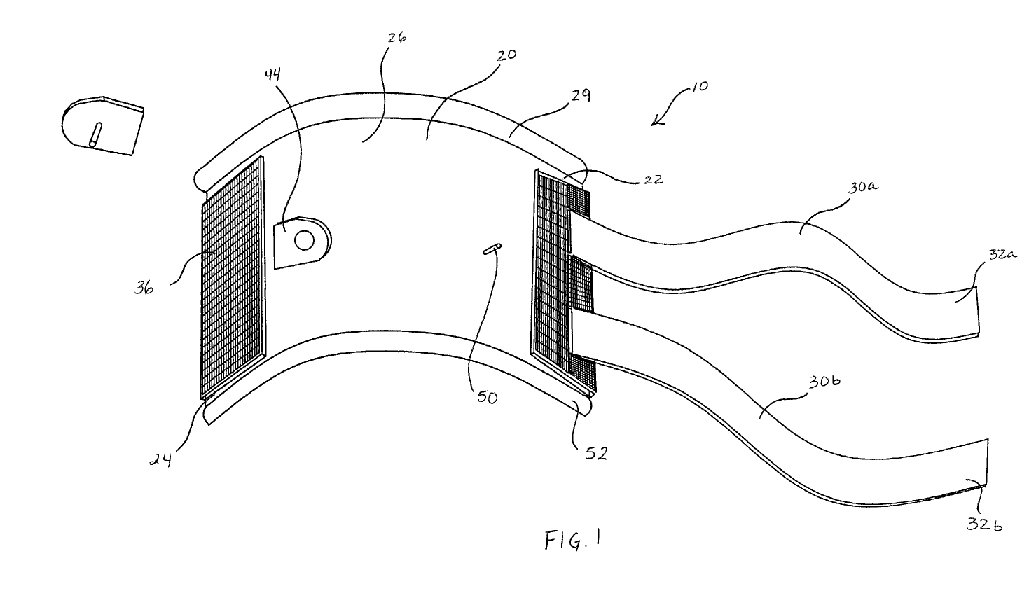

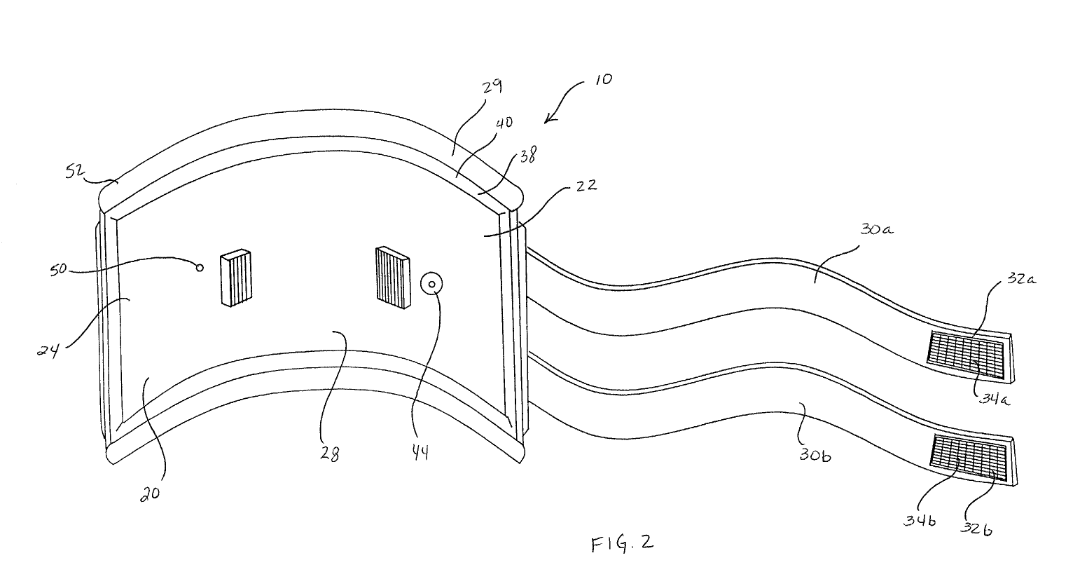

Referring now to FIG. 1 and FIG. 2, the present invention is a sleep apnea device 10, wherein support member 20 has a generally arcuate configuration such that the arcuate configuration enables generally flush placement of sleep apnea device 10 against the neck of a user. Preferably, first and second straps 30a and 30b, respectively, are attached proximate to first end 22 of support member 20 as more fully described below. Although the preferred embodiment includes two straps, 30a and 30b, one skilled in the art would readily recognize that a single strap or more than two straps could be utilized, wherein secure positioning of sleep apnea device 10 around the neck of a user could be effectively maintained.

Preferably, distal ends 32a and 32b of first and second straps 30a and 30b, respectively, have strap fastening means 34a and 34b, respectively, engaged therewith, wherein strap fastening means 34a and 34b is preferably hook and loop fastener. Preferably, second end 24 of support member 20 has cooperative fastening means 36 provided thereon, wherein strap fastening means 34a and 34b securely, adjustably and removably engages therewith. Preferably, cooperative fastening means 36 is hook and loop fastener, however, one skilled in the art would readily recognize that other types of cooperative fastening means could be utilized such as, for exemplary purposes only, buckles, snaps, magnets, clasps or loops.

Support member 20 has outer surface 26, inner surface 28 and peripheral edge 29. Preferably, peripheral edge 29 is substantially covered with a flexible, comfortable cushioning material 52, whereby user comfort is maximized during the wearing of device 10. Seal 38 is preferably positioned on inner surface 28 proximate to peripheral edge 29, wherein seal 38 is preferably rubber gasket 40, capable of forming a substantially airtight bond with the neck of a user. Although it is preferred that seal 38 is rubber gasket 40, it is anticipated that other types of seals or materials could be utilized such as, for exemplary purposes only, plastic, shaped foam or any other plastic or rubber coated or otherwise air-impervious material.

The preferred positioning of seal 38 proximate to peripheral edge 29 of device 10 enables formation of a substantially airtight zone 42 between inner surface 22 of device 10 and the neck of a user. Preferably, valve 44 is provided on support member 20, wherein valve 44 is configured to permit one-way passage of air from airtight zone 42, thus enabling the creation of a negative pressure or vacuum between inner surface 22 of device 10 and the neck of a user, that is, within airtight zone 42.

Preferably, the degree of curvature of support member 20 is provided to conform with the degree of curvature of the outer surface of the neck of a user, wherein alternately dimensioned devices 10 are anticipated to most suitably conform to a variety of individual neck sizes and shapes. Moreover, support member 20 may be manufactured from suitably resilient material to enable individual adjustment and shaping thereof. Preferably, the dimensions of support member 20 enable comfortably secure placement of device 10 on the outer surface of a user's neck, generally along the mandibular area and on the clavicular area, wherein support member 20 preferably does not exert pressure upon the carotid body or any other sensitive structure.

In the preferred embodiment, data collection valve 50 is provided, wherein a data collection vessel (not shown) or device may be connected thereto for analytical analysis of the performance of device 10. Preferably, data collection valve 50 enables connection to a test tube (not shown), wherein measurement and recording of water pressure can be correlated to effective performance of device 10.

In an alternate embodiment, support member 20 could have a generally flat, flexible configuration, wherein manipulatably flush placement of sleep apnea device 10 against the neck of a user could be enabled.

In an alternate embodiment, support member 20 could be formed from multiple linked sections, wherein a substantially airtight and flexible seal could be provided at the linkage thereof.

In an alternate embodiment, device 10 could have a hinged positioning system in lieu of straps.

In an alternate embodiment, support member 20 could carry an integrally formed fastening means.

In an alternate embodiment, device 10 could have any number of straps.

In an alternate embodiment, device 10 could have a quick-release feature, thereby enabling immediate removal of support member 20 from the neck of a user if necessary.

In an alternate embodiment, more than one vacuum valve could be provided for sleep apnea device 10.

In an alternate embodiment, sleep apnea device 10 could be manufactured and utilized without data collection valve 50.

In an alternate embodiment, sleep apnea device 10 could be fitted with electronic data collection instrumentation known in the art.

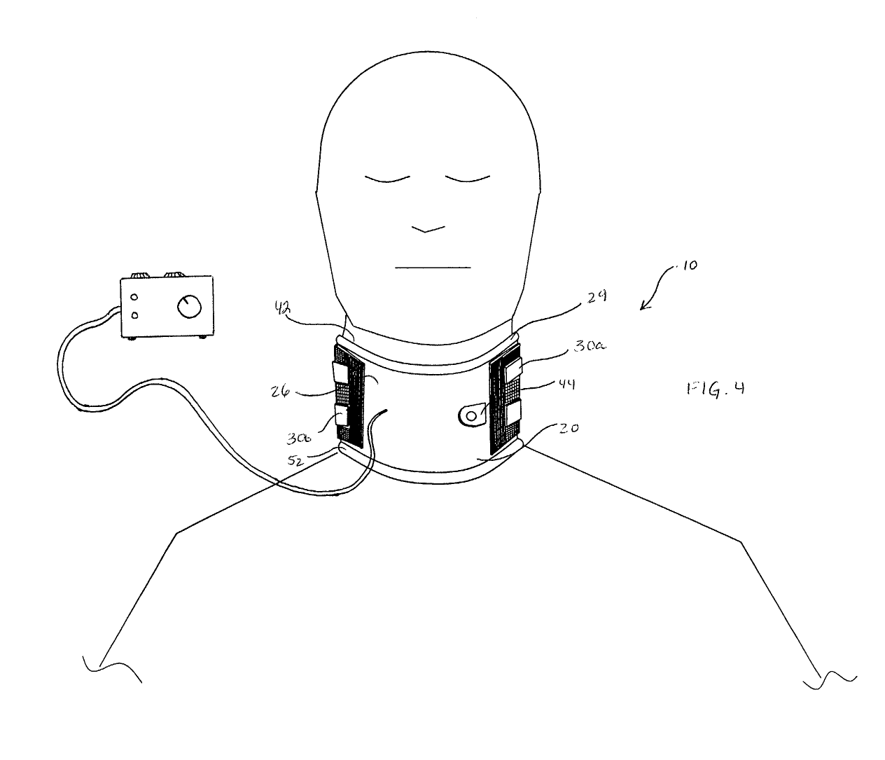

In an alternate embodiment, as shown in FIG. 4, an external negative pressure means such as, for exemplary purposes only, a vacuum or air pump, could be connected to valve 50 or valve 44 of sleep apnea device 10 to control and/or assist in the creation and/or maintenance of negative pressure.

In use, sleep apnea device 10 is placed with inner surface 22 of support member 20 facing the outer surface of the front of the neck of a user, wherein preferred placement on the neck is along the mandibular area and on the clavicular area, wherein support member 20 preferably does not exert pressure on the carotid body or any other sensitive structure. With support member 20 generally proximate to the neck, straps 30a and 30b are secured whereby seal 40 is substantially flush with the skin and substantially airtight zone 42 is formed between inner surface 22 thereof and the neck of a user.

As the neck of the user rises and falls during normal breathing rhythm, valve 44 allows the escape of air from airtight zone 42, such that the respiratory motion of the soft structures of the neck provides the energy to create the vacuum or negative pressure within airtight zone 42, whereby the self-regulating system is able to limit subsequent movement of the soft structures of the neck. As a user's breathing pattern becomes interrupted by apneic episodes, or as respiration becomes more urgent or labored, the negative pressure or vacuum increases, thereby effectively drawing open the air passages of a user as needed. The self-regulating pressure essentially prevents the closure of the air pathways, enabling a user to counteract the bothersome and potentially detrimental effects of sleep apnea as needed, instead of in response to a preset pressure or mechanism. That is, as the neck of the user expands, valve 44 enables the passage of air from airtight zone 42 thus creating a negative pressure or vacuum within zone 42 when the neck of the user recedes in inhalation. As breathing becomes apneic, respiratory movements become greater, thus increasing the vacuum and effectively holding the airways open. The pressure gradient within zone 42 increases or decrease with the need for it, thus sleep apnea device 10 and method thereof automatically adjusts to the respiratory need of the user.

Having thus described exemplary embodiments of the present invention, it should be noted by those skilled in the art that the within disclosures are exemplary only, and that various other alternatives, adaptations, and modifications may be made within the scope of the present invention. Accordingly, the present invention is not limited to the specific embodiments illustrated herein, but is limited only by the following claims.

What is claimed is:

1. An air pathway clearance device for wear on the neck of a user, comprising:
a support member, said support member having an inner surface, an outer surface and a peripheral edge;
securing means, said securing means carried by said support member and said securing means enabling retention of said support member on the neck of the user;
retention means, said retention means enabling retention of said securing means relative to said support member;
sealing means, said sealing means carried by said support member proximate to said inner surface of said support member, said sealing means enabling substantially airtight positioning of said support member against the neck of the user, and said sealing means generally protruding from said inner surface of said support member, thereby defining an air compartment between said support member and the neck of a user;
at least one valve, said valve carried by said support member, wherein said valve enables the exit of air from said air compartment.

2. The air pathway clearance device of claim 1, wherein said support member is generally arcuately shaped and is formed from substantially rigid material.

3. The air pathway clearance device of claim 2, wherein said arcuate shape of said support member is adjustable.

4. The air pathway clearance device of claim 1, wherein said support member is formed from substantially flexible material.

5. The air pathway clearance device of claim 1, wherein said securing means is at least one strap and said retention means is hook and loop fastener.

6. The air pathway clearance device of claim 1, wherein said securing means is at least one strap and said retention means is at least one buckle.

7. The air pathway clearance device of claim 1, wherein said securing means is at least one strap and said retention means is at least one snap.

8. The air pathway clearance device of claim 1, wherein said securing means is at least one strap and said retention means is at least clasp.

9. The air pathway clearance device of claim 1, wherein said securing means is at least one strap and said retention means is at least one magnet.

10. The air pathway clearance device of claim 1, further comprising a cushioning material carried by said support member.

11. The air pathway clearance device of claim 10, wherein said cushioning material substantially surrounds said peripheral edge of said support member.

12. The air pathway clearance device of claim 1, wherein said sealing means is a rubber gasket.

13. The air pathway clearance device of claim 1, further comprising a means for measuring performance.

14. The air pathway clearance device of claim 13, wherein said means for measuring performance is a valve.

15. The air pathway clearance device of claim 13, wherein said means for measuring performance is electronic data collection instrumentation.

16. The air pathway clearance device of claim 1, wherein said support member is formed from a plurality of linked segments.

17. A sleep apnea device for use on the neck of a user, comprising:
a neck cuff having a generally arcuate shape, a concave surface and a peripheral edge;
at least one strap carried by said neck cuff, said at least one strap having at least one strap fastener for securing the position of said neck cuff on the neck of the user;
a gasket, said gasket carried proximate to said concave surface of said neck cuff;
at least one vacuum regulator, said vacuum regulator carried by said neck cuff, wherein said vacuum regulator permits a unidirectional flow of air.

18. The sleep apnea device of claim 17, further comprising a generally resilient border substantially covering said peripheral edge of said neck cuff.

19. The sleep apnea device of claim 18, further comprising a data collection port.

20. The sleep apnea device of claim 17, further comprising a pressure generating device, said pressure generating device assisting the unidirectional flow of air through said at least one vacuum regulator.

21. The method of treating sleep apnea comprising the steps of:
a. obtaining a sleep apnea device having a support member with an inner surface, at least one strap carried by said support member, at least one gasket carried proximate to said inner surface of said support member, and at least one valve carried by said support member;
b. placing said sleep apnea device on the neck of a user with said inner surface of said support member proximate to the outer surface of the neck of a user;
c. securing said at least one strap around the neck of a user;
d. forming a generally airtight seal between said gasket and the neck of a user, thereby defining a substantially airtight zone;
e. creating a negative pressure within said substantially airtight zone;
f. utilizing a vacuum resulting from said negative pressure to maintain open air passages for the user.

22. The method of treating sleep apnea of claim 21, wherein said negative pressure is created by allowing air to escape from said substantially airtight zone through said valve, in self-regulated response to respiratory movement of the soft tissues of the neck of the user.

23. The method of treating sleep apnea of claim 21, wherein said placement of said support member on the neck of a user is along the mandibular area and on the clavicular area.

24. The method of treating sleep apnea of claim 23, wherein said support member extends over the frontal neck of the user without exerting pressure on the carotid body.
